(19) United States
(12) Patent Application Publication
(10) Pub. No.: US 2006/0253058 A1
(43) Pub. Date: Nov. 9, 2006

(54) Weft-inserted elastic adhesive bandage and method

(76) Inventor: John C. Evans, Nr Rochdale (GB)

Correspondence Address: ADAMS EVANS P.A., 301 SOUTH TRYON STREET, SUITE 2180, TWO WACHOVIA CENTER, CHARLOTTE, NC, 28282-1991, US

(21) Appl. No.: 10/561,359

(22) PCT Filed: Jun. 10, 2003
(86) PCT No.: PCT/US03/18326

§ 371 Date: Apr. 27, 2006

(51) Int. Cl. A61F 15/00

(52) U.S. Cl. 602/41

(57) ABSTRACT

An elastic bandage including an elongate warp-knitted fabric substrate including a lock stitch formed in opposing side edges of the fabric substrate to prevent fraying. The substrate includes a cotton and elastic yarns in a warp direction and weft-inserted yarns in weft direction. The weft-inserted yarns include cotton, with an adhesive coated onto or impregnated into the substrate.

### TECHNICAL FIELD AND BACKGROUND OF THE INVENTION

This invention relates generally to the field of orthopedic medicine and more specifically to a medical bandaging product and material comprising a fabric substrate with weft-inserted yarns and method of constructing such bandages.

Conventional elastic adhesive bandages (EAB's) are used in the support, aid and treatment of sprains, strains and injuries, and are required to be constructed with sufficient elasticity to conform to varying body contours and to ensure limited movement. These EAB's generally include a pressure-sensitive adhesive layer. Bandages of this type, in particular, woven bandages, are disclosed in U.S. Pat. No. 6,267,744.

Conventional EAB's are generally constructed by incorporating cotton fibers, typically 1⅛-1¼ staple length, that have been mechanically or chemically crimped to provide stretch. Stretch and recovery properties of crimped yarns in fabric are the most important parameters controlling performance of the stretch fabric. The requisite stretch is generally obtained by applying an S or Z twist to a two-ply cotton yarn at about 1900-2300 turns per meter. The yarn is then wet twisted to provide shrink and recovery. Furthermore, an increase in yarn twist up to an optimum level increases both yarn extensibility and recovery. Mechanically crimped cotton yarns, once treated in certain aqueous solutions, swell and shrink. When these yarns are relaxed they attempt to return to the highly twisted position at which they were curled. Examining this state reveals that the yarn sustains greater than 100% stretch. The fiber properties of the cotton maturity and staple length may greatly affect the performance of spun and plied yarns in stretch fabrics, especially with regard to single and plied yarns.

Single yarns appear to have greater maximum shrinkage potential such that properties of the yarn, in conjunction with fabric shrinkage, present great potential for fabric stretchability. Woven or knitted elastic bandages made of such yarns exhibit good stress retention properties.

Although conventional methods of using highly twisted cotton yarns provide an adequate bandage for use in the area of retention and support, the manufacture of such EAB's can be very expensive because of the numerous steps that must be carried out. These steps include warping, weaving, finishing, compaction and wet processing, crimping drying and ironing. The stretch and recovery properties of the prior art fabrics are also difficult to sustain over time.

Conventional EAB's known in the art also include ravel-resistant side edges and may include an adhesive on one or both major surfaces so as to be self-adhesive or adhesive to the skin of the wearer. One major importance of using an adhesive has been that it can reduce the slippage of the wrapped compression bandage while the patient moves, thereby providing more uniform support and compression for extended periods of time. Adhesive applied to one side of the fabric allows the bandage to remain fixed in place.

The disadvantages found in conventional EAB's, however, are observed in the fatigue of properties, roughness of the surface of the fabric, limited conformability and costs associated with manufacture.

### SUMMARY OF THE INVENTION

Therefore, it is an object of the invention to provide a weft-inserted bandage having elastic and inelastic warp and weft-inserted yarns coated on one side with a pressure sensitive adhesive (PSA) to allow the bandage to remain fixed in place.

It is another object of the invention to provide a medical bandaging product including a medical bandaging material formed from a weft-insertion technology utilizing both elastic and inelastic yarns that do not lose elasticity as readily as conventional EAB's.

It is another object of the present invention to provide a bandage having a weft-insertion structure that utilizes the elastomeric properties in a manner that offers improved conformability and is softer than conventional EAB's.

It is another object of the invention to provide a medical bandaging product that can be manufactured at a less expensive cost than that of conventional EAB's.

It is another object of the invention to provide a medical bandaging material that includes an adhesive-coated substrate formed from knitted cotton yarns and elastic yarns.

These and other objects of the present invention are achieved in the preferred embodiments disclosed below by providing an elastic bandage, comprising an elongate warp-knitted fabric substrate including a lock stitch formed in opposing side edges of the fabric substrate to prevent fraying, the substrate comprising cotton and elastic yarns in a warp direction, the substrate comprising weft-inserted yarns in weft direction, the weft-inserted yarns comprising cotton, and an adhesive coated onto or impregnated into the substrate.

According to one preferred embodiment of the invention, the cotton yarns comprise at least 97 percent of the total knitted weight of the fabric substrate.

According to another preferred embodiment of the invention, the warp yarns are selected from the group consisting of single ply yarns and double ply yarns.

According to yet another preferred embodiment of the invention, the weft-inserted yarns are between 600-800 Decitex.

According to yet another preferred embodiment of the invention, the lock stitch is formed of polyester yarn.

According to yet another preferred embodiment of the invention, the weight of the fabric substrate is between 200-250 grams per square meter.

According to yet another preferred embodiment of the invention, the weight of the fabric substrate is 230 grams per square meter.

According to yet another preferred embodiment of the invention, the elastic yarns comprise a manufactured fiber in which the fiber forming substance is a long-chain synthetic polymer comprised of at least 85% of a segmented polyurethane.

According to yet another preferred embodiment of the invention, the bandage has stretch of between 85-95 percent.

According to yet another preferred embodiment of the invention, the bandage has a regain of 50-60 percent.

According to yet another preferred embodiment of the invention, the adhesive comprises a pressure-sensitive adhesive.

According to yet another preferred embodiment of the invention, said adhesive comprises a coadhesive, which may be an latex or non-latex adhesive.

According to yet another preferred embodiment of the invention, the adhesive is coated onto one side of the fabric substrate.

According to yet another preferred embodiment of the invention, an elastic bandage is provided, comprising an elongate warp-knitted fabric substrate including a polyester lock stitch formed in opposing side edges of the fabric substrate to prevent fraying. The substrate comprises cotton yarns and elastic yarns in a warp direction and weft-inserted yarns in weft direction, the weft-inserted yarns comprising cotton. A coadhesive is coated onto one side of the substrate.

According to yet another preferred embodiment of the invention, the cotton yarns define a cross-sectional area greater than the elastic yarns.

According to yet another preferred embodiment of the invention, the fabric substrate includes an elastic yarn and a pair of cotton yarns lying on respective opposing sides of the elastic yarn to cover and inclose the elastic yarn within a layer of inelastic yarns.

According to yet another preferred embodiment of the invention, the pair of cotton yarns have opposite twist directions so that adjacent cotton yarns lying on opposite sides of adjacent elastic yarns have the same direction of twist.

According to yet another preferred embodiment of the invention, the adhesive is blown onto the fabric substrate to form a discontinuous adhesive layer to allow the bandage to breathe.

### BRIEF DESCRIPTION OF THE DRAWINGS

Some of the objects of the invention have been set forth above. Other objects and advantages of the invention will appear as the invention proceeds when taken in conjunction with the following drawings, in which:

FIG. 1 is a perspective view of an elastic bandage according to an embodiment of the invention; and

FIG. 2 is stitch pattern of the fabric structure of the elastic bandage.

### DESCRIPTION OF THE PREFERRED EMBODIMENT AND BEST MODE

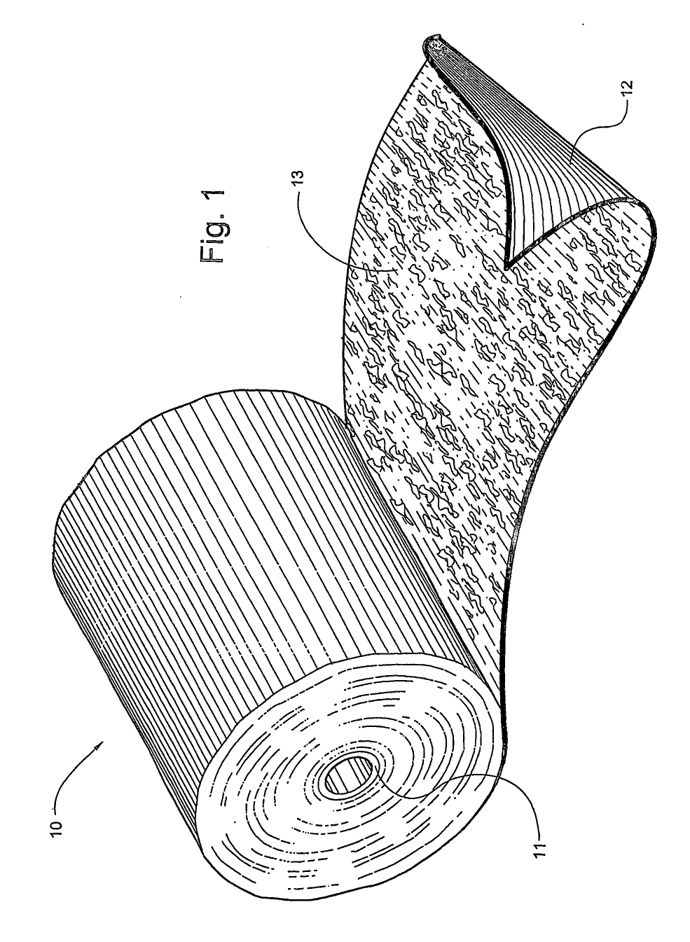

Referring now specifically to the drawings, an elastic bandage according to the present invention is illustrated in FIG. 1 and shown generally at reference numeral 10. The bandage 10 is wound onto a core 11 to form a roll. The bandage 10 includes a warp-knitted substrate 12 with an adhesive 13 coated onto one side. The adhesive 13, which may be a coadhesive, i.e., it adheres to itself but not as well to other substances, provides sufficient adherence to maintain the integrity of the roll. Appropriate amounts are dispensed as needed by pulling a length of bandage 10 off of the roll and severing the length from the bandage 10 remaining on the core 11. The removed length of bandage 10 is then applied to a limb in a conventional manner.

The fabric substrate 12 is a compression support fabric comprising a weft-inserted substrate constructed such that elasticized and inelastic yarns are arranged in an alternating elastic and non-elastic pair, woven in the form of a chain stitch construction with a cotton yarn passing across the rows of chain stitches joining them together. The construction of the bandage has a cross-sectional area wherein the exposed surface area of the inelastic yarns is greater than that of the elastic yarns.

This construction is accomplished by laying on each side of the elastic yarn an inelastic yarn, preferably cotton. The cotton yarns preferably have opposing twist, ‘S’ or ‘Z’ direction, or of the same direction, all ‘S’ or alternatively all ‘Z’, such that adjacent inelastic yarns are always of the same direction. This cross-sectional construction results in covering and inclosing the elasticized yarn within the inelastic yarns.

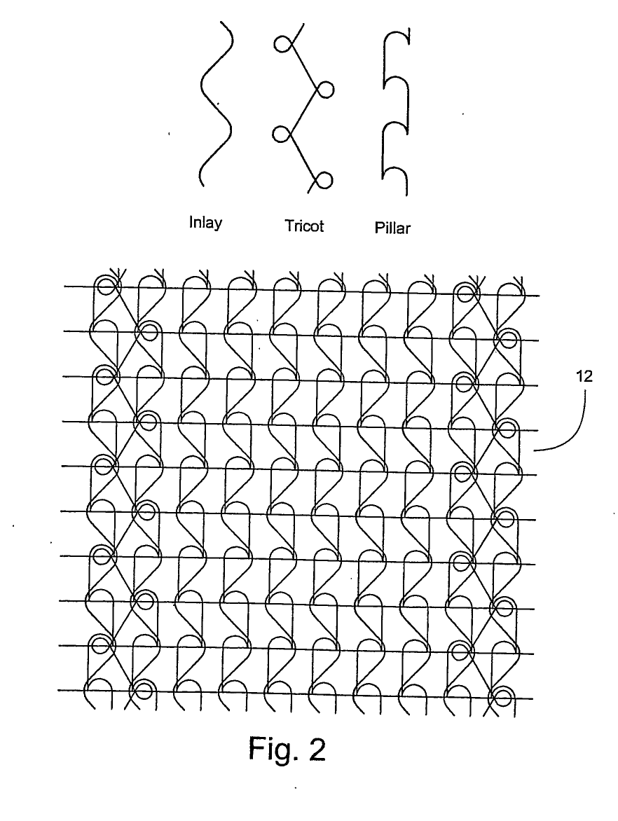

The stitch diagram of a preferred embodiment of the substrate 12 is shown in FIG. 2.

In one embodiment of the invention, the bandage 10 is constructed in such a manner that achieves a soft, elastic appearance to the fabric. Although the elastic yarns may be incorporated in any suitable direction along the substrate, the elastic yarns are preferably incorporated along the length of the substrate, which permits the substrate to be stretched and extended in the lengthwise direction while maintaining substantial widthwise integrity. Suitable elastic yarns may include, but are not limited to, those formed from rubber or elastomeric polymers which have high extendibility and exhibit substantially complete and rapid elastic recovery. Such elastomer-based yarns may be mono- or multi-filamentary in nature, such as Spandex. Spandex is a manufactured fiber in which the fiber forming substance is a long-chain synthetic polymer comprised of at least 85% of a segmented polyurethane. The polymer chain is a segmented block copolymer containing long, randomly coiled, liquid, soft segments that move to a more linear, lower entropy, structure. The hard segments act as “virtual cross-links” that tie all the polymer chains together into an infinite network. This network prevents the polymer chains from slipping past each other and taking on a permanent set or draw. When the stretching force is removed, the linear, low entropy, soft segments move back to the preferred randomly coiled, higher entropy state, causing the fiber to recover to its original shape and length. This segmented block copolymer is formed in a multi-step proprietary process. It is extruded into a fiber as a monofilament threadline or for most products into a multiplicity of fine filaments that are coalesced shortly after they are formed into a single threadline.

The inelastic yarns are incorporated into the pillar stitch of the substrate 12 of the bandage 10 in the count range of 250-300 Decitex. The use of such inelastic yarns and the method described herein generally leads to qualities and characteristics particularly good for the manufacture of elastic adhesive bandages (EAB's).

The cotton yarns used in the inlay stitch will be in a count range of 500-800 Decitex. Also, the cotton yarn preferably has a tenacity of 15 g/tex. Furthermore, while the cotton yarns may have any extension, the preferable extension (elongation) is between 8-30%.

The substrate 12 of the present invention is preferably a weft-inserted substrate incorporating a tricot stitch over the last two threads of the bandage 10 to render the product non-fray, and is knitted on a knitting machine employing 3-4 guide bars.

The substrate may have any suitable thickness, more specifically, the thickness is 1 mm to 1.3 mm and any weight per unit area with a ratio of inelastic yarns to elastic yarns of 1:1. Although the fabric of the substrate is preferably a weft-inserted fabric, many suitable knits may be utilized.

The adhesive used in the embodiment of the present invention may be of a pressure sensitive type, which will satisfy the functional requirements of the medical bandage. The high adhesive properties of the bandage are capable of withstanding stress of prolonged or strenuous activity. The adhesive is preferably blown onto one surface of the substrate 12. The entrained air forms voids on the surface of the substrate 12 that form discontinuous uncoated areas that permit air to pass through the bandage 10.

In a preferred embodiment of the invention, the relaxed courses range from 110-130 per 10 cm and between 40-70% stretched courses, but preferably 50-75 per 10 cm. Stretch is between 70-100% and regain is between 40-70%.

According to another preferred embodiment of the invention, the fabric material is formed from elastomeric yarn of between 70-150 Decitex and may include an inlay yarn of between 500-800 Decitex. Preferably, the cotton yarn employed in the substrate 12 has a modulus of 15 g/tex.

According to another preferred embodiment of the invention, the extension (elongation) of the cotton yarns employed in the fabric material is between 6-15% and the knitted fabric material exhibits an extensibility of 50-80% prior to the application of the adhesive.

According to another preferred embodiment the cotton yarns used in the substrate are two fold cottons or single cotton yarns with a count range of 180 to 220 Decitex.

One example of the bandage is as follows:

176-240 inch Karl Mayer warp knitting machine fitted
with 12-24 feeders with separate feed mechanism

| | |
|---|---|
| Warp (pillar) yarn | 2 fold 60's ne cotton yarn |
| weft yarn | single 8's cotton yarn |
| Elastic Yarn | Spandex 156 Decitex |
| Adhesive | E3 Rubber-based adhesive applied at a rate of |
| | 40 percent of fabric weight. |
| Fabric Weight | 243 grams per square meter |

Alternatively, the adhesive may be a synthetic polymer adhesive, such as a neoprene adhesive.

A medical bandage is described above. Various details of the invention may be changed without departing from its scope. Furthermore, the foregoing description of the preferred embodiment of the invention and the best mode for practicing the invention are provided for the purpose of illustration only and not for the purpose of limitation—the present invention being defined by the claims.

I claim:

1. An elastic bandage, comprising:
(a) an elongate warp-knitted fabric substrate including a lock stitch formed in opposing side edges of the fabric substrate to prevent fraying;
(b) the substrate comprising cotton and elastic yarns in a warp direction;
(c) the substrate comprising weft-inserted yarns in weft direction, the weft-inserted yarns comprising cotton; and
(d) an adhesive coated onto or impregnated into the substrate.

2. An elastic bandage according to claim 1, wherein the cotton yarns comprise at least 97 percent of the total knitted weight of the fabric substrate.

3. An elastic bandage according to claim 1, wherein the warp yarns are selected from the group consisting of single ply yarns and double ply yarns.

4. An elastic bandage according to claim 1, wherein the weft-inserted yarns are between 600-800 Decitex.

5. An elastic bandage according to claim 1, wherein the lock stitch is formed of polyester yarn.

6. An elastic bandage according to claim 1, wherein the weight of the fabric substrate is between 200-250 grams per square meter.

7. An elastic bandage according to claim 1, wherein the weight of the fabric substrate is 230 grams per square meter.

8. An elastic bandage according to claim 1, wherein the elastic yarns comprise a manufactured fiber in which the fiber forming substance is a long-chain synthetic polymer comprised of at least 85% of a segmented polyurethane.

9. An elastic bandage according to claim 1, wherein the bandage has stretch of between 85-95 percent.

10. An elastic bandage according to claim 1, wherein the bandage has a regain of 50-60 percent.

11. An elastic bandage according to claim 1, wherein said adhesive comprises a pressure-sensitive adhesive.

12. An elastic bandage according to claim 1, wherein said adhesive comprises a coadhesive.

13. An elastic bandage according to claim 1, wherein said adhesive is discontinuously applied onto one side of the fabric substrate to thereby leave exposed areas of the substrate for the passage of air therethrough.

14. An elastic bandage, comprising:
(a) an elongate warp-knitted fabric substrate including a lock stitch formed in opposing side edges of the fabric substrate to prevent fraying;
(b) the substrate comprising cotton yarns and elastic yarns in a warp direction;
(c) the substrate comprising weft-inserted yarns in weft direction, the weft-inserted yarns comprising cotton; and
(d) a coadhesive coated onto one side of the substrate.

15. An elastic bandage according to claim 14, wherein the cotton yarns define a cross-sectional area greater than the elastic yarns.

16. An elastic bandage according to claim 14, wherein the fabric substrate includes an elastic yarn and a pair of cotton yarns lying on respective opposing sides of the elastic yarn to cover and inclose the elastic yarn within a layer of inelastic yarns.

17. An elastic bandage according to claim 16, wherein the pair of cotton yarns have opposite twist directions so that adjacent cotton yarns lying on opposite sides of adjacent elastic yarns have the same direction of twist.

18. An elastic bandage according to claim 14, wherein the adhesive is blown onto the fabric substrate to form a discontinuous adhesive layer to allow the bandage to breathe.

19. A method of forming an elastic bandage, comprising the steps of:
(a) warp-knitting an elongate fabric substrate including a lock stitch formed in opposing side edges of the fabric substrate to prevent fraying, the substrate comprising cotton yarns and elastic yarns in the warp direction and weft-inserted cotton yarns in the weft direction;
(b) applying an adhesive onto one side of the substrate; and
(c) forming the substrate with the adhesive thereon into a roll.

20. A method according to claim 19, wherein the step of applying the adhesive comprises the step of applying a coadhesive discontinuously to the substrate to permit passage of air through the bandage.
